(19) United States
(12) Patent Application Publication
(10) Pub. No.: US 2015/0168372 A1
(43) Pub. Date: Jun. 18, 2015

(54) Method And Apparatus For Quantifying Oxygen Consumption Of Mitochondria

(71) Applicant: The Trustees of Dartmouth College, Hanover, NH (US)

(72) Inventor: Roger Springett, Hanover, NH (US)

(73) Assignee: The Trustee of Dartmouth College, Hanover, NH (US)

(21) Appl. No.: 14/400,722

(22) PCT Filed: Apr. 12, 2013
(86) PCT No.: PCT/US2013/036462

§ 371 Date: Nov. 12, 2014

Related U.S. Application Data
(60) Provisional application No. 61/623,329, filed on Apr. 12, 2012.

(51) Int. Cl. G01N 33/497; G01N 33/487; G01N 33/00; G01N 7/00

(52) U.S. Cl. CPC G01N 33/497; G01N 7/00; G01N 33/487; G01N 33/0036

(57) ABSTRACT

A system for measuring oxygen consumption uses a chamber holding culture medium with a biological material. A resistively gas-permeable membrane has a first side contacting culture medium in the chamber and a first sensor for measuring oxygen in the culture medium. A second sensor measures oxygen in gas adjacent a second side of the membrane. The sensors provide data to a processor having a memory with machine readable instructions for determining oxygen consumption from the data. Alternatively, a method of measuring oxygen flux begins with placing biological material, with fluid, in the chamber; exposing the fluid to a resistively-permeable membrane with oxygen-containing gas on a second side of the membrane; measuring oxygen in the fluid; measuring oxygen in the gas, and calculating the oxygen flux of the biological material from an oxygen permeability constant of the membrane and a difference between the oxygen concentrations of the fluid and gas.

### FEDERAL INTEREST

The United States Government has certain rights in the invention. This invention was made with government support under 5R21RR25803 awarded by the National Institute of Health. The government has certain rights in the invention.

### FIELD

The present document relates to the field of instrumentation for biomedical research. In particular, the document relates to the field of instruments for measuring uptake and/or production of oxygen by biological systems, including systems containing eukaryotic cells, prokaryote cells or mitochondria in-vitro and in-vivo.

### BACKGROUND

Mitochondria are subcellular organelles found in eukaryotic cells, including human cells. These organelles have their own DNA and ribosomes, together with an inner membrane typically folded in cristae. This inner membrane has embedded a variety of proteins, together with bound cofactors and cytochromes, that together perform oxidative metabolism and synthesis of the majority of cellular adenosine triphosphate (ATP) in most eukaryotes. The ATP generated by mitochondria is critical for survival and reproduction of most eukaryotic cells as energy released by dephosphorylization of ATP powers most cellular synthetic activity and muscle contraction.

Oxidative metabolism performed in the mitochondria includes beta-oxidation of fatty acids to produce FADH, NADH, and acetyl-coenzyme A (Acetyl-COA), oxidation of pyruvate to acetyl-COA and NADH, and the Krebs cycle where acetyl-coenzyme A is oxidized to produce NADH (Nicotinamide Adenine Dinucleotide hydride) and FADH (flavin adenine dinucleotide dihydride). The NADH and FADH in turn become substrates for the proteins of the electron transport chain (ETC) and cytochromes on the inner mitochondrial membrane that generates an electrochemical gradient across the membrane; the NADH and FADH are oxidized by the ETC to NAD⁺ (Nicotinamide adenine dinucleotide) and FAD⁺ (flavin adenine dinucleotide). This electrochemical gradient is in turn harnessed to produce ATP by adding a third phosphate to adenosine diphosphate (ADP). These reactions are well understood and will not be detailed here.

Mitochondria of different mammalian species are known to operate with varying efficiency and varying production of stray free radicals; these free radicals are suspected to be involved with aging. A number of mitochondrial diseases are known, where genetic anomalies of either the mitochondrial or cellular DNA result in accumulations of malfunctioning proteins that may not properly function; these diseases may result in reduced ATP production under some conditions, or may result in accumulation of toxic byproducts. Some poisons or drugs, including carbon monoxide (CO) and cyanide (CN), are known that act directly in the mitochondria, and others act indirectly by regulating production of some mitochondrial proteins, or even regulating production of mitochondria. Alterations in mitochondrial function have been implicated in Alzheimer's disease, Parkinson's disease, amyotrophic lateral sclerosis (ALS) and diabetes. Study of mitochondrial function, and factors that can alter such function, are therefore of interest to biologists and medical researchers who wish to better understand physiology and pathophysiology, and who wish to determine how better to treat some of these diseases.

Some eukaryotic cells also have chloroplasts capable of performing photosynthesis, these cells may release oxygen when illuminated. For purposes of this document, oxygen flux of a biological material is the consumption or release of oxygen by that material.

Some prokaryotic cells may also consume, or release, oxygen; consumption and release of oxygen by these cells, and the factors that increase or decrease such consumption and release, may also warrant study.

### SUMMARY

A system for measuring oxygen consumption in mitochondria uses a chamber adapted to contain a culture medium containing a biological material. A resistively gas-permeable membrane has a first side positioned to contact culture medium present in the chamber and a first sensor positioned to measure oxygen in culture medium in the chamber. A second sensor is positioned to measure oxygen in gas adjacent a second side of the membrane. The sensors are coupled to provide data to a processor, the processor has a memory having recorded therein machine readable instructions for determining oxygen consumption by the biological material from the data.

A method of measuring oxygen flux of a biological material begins with placing the material, with a fluid, in a chamber; exposing the fluid to a first side of a resistively-permeable membrane; exposing a second side of the resistively-permeable membrane to a gas mixture, the gas mixture containing oxygen at a gas oxygen concentration; measuring an oxygen partial pressure of the fluid; and calculating the oxygen flux of the biological material from an oxygen permeability constant of the membrane and a difference between the oxygen concentration of the fluid and gas oxygen concentration.

### BRIEF DESCRIPTION OF THE FIGURES

FIG. 1 is a summary block diagram illustrating a system for measuring oxygen consumption rates of suspended mitochondria or living cells.

FIG. 2 is a schematic cross section of a sample chamber for use in the system of FIG. 1.

FIG. 3 is a schematic cross section of an alternative sample chamber for use with suspended cells.

FIG. 4 is a circuit diagram of an electrical circuit roughly analogous to a chamber with mitochondria and chamber oxygenation tube.

FIG. 5 is an approximate flowchart of a method of using the apparatus of FIG. 1-3 to measure oxygen consumption in a biological material.

### DETAILED DESCRIPTION OF THE EMBODIMENTS

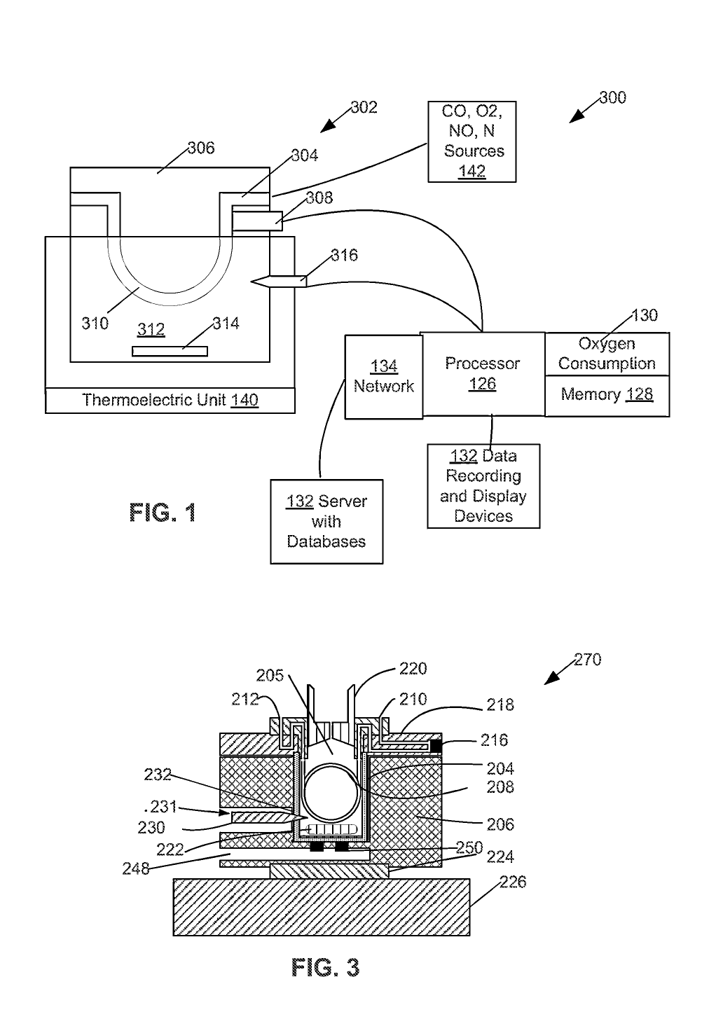

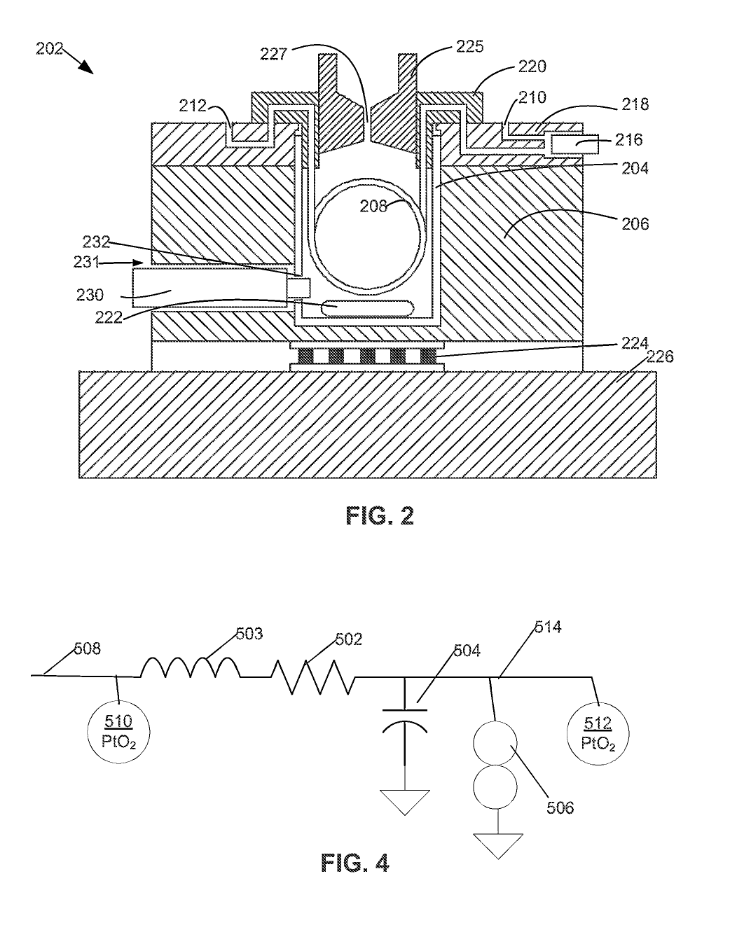

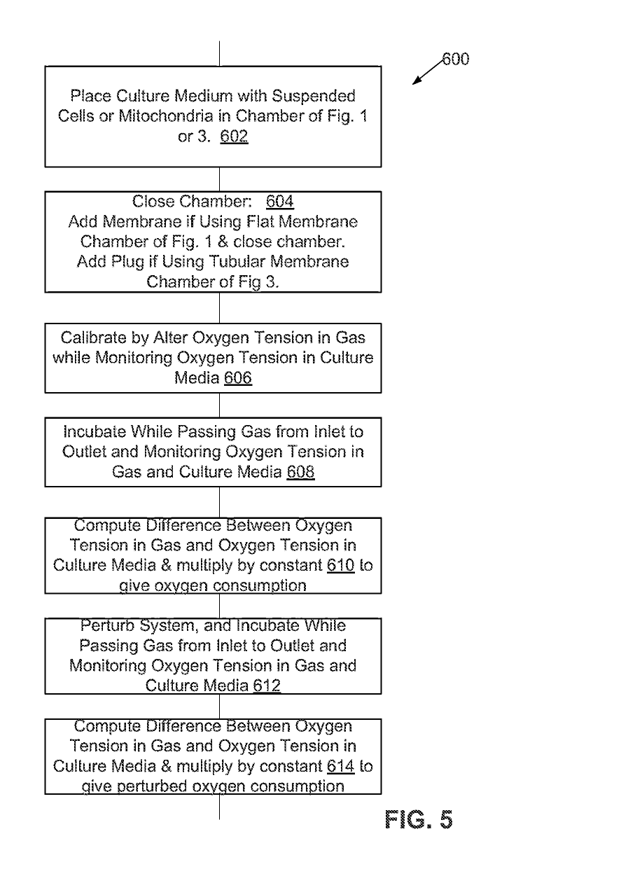

A system 300 for measuring oxygen consumption or production by a biological material, in an embodiment the biological material includes suspended mitochondria or suspended eukaryotic cells containing mitochondria, is illustrated in FIG. 1. In alternative embodiments, the biological material may be a suspension of aerobic bacteria, facultative anaerobic bacteria, or cyanobacteria capable of performing oxidative metabolism System 300 has a processor 126 having a memory subsystem 128; memory subsystem 128 includes machine readable instructions for determining oxygen consumption by cells or mitochondria suspended in a chamber 302. Chamber 302 may be the suspended-cell chamber illustrated in FIG. 2 or 3, and incorporates the elements illustrated in FIG. 1. Processor 126 is also coupled to data recording and display devices 132, and through a network interface 134 and a network to a network server 132 that may contain a database of data recorded by system 300. In particular embodiments, the cells or mitochondria are suspended in a culture medium that is capable of supporting most if not all metabolic functions of the cells or mitochondria.

Chamber 302 is coupled to receive a gas mixture from gas sources 142. This gas mixture is coupled to pass through inlet passage 304 in lid 306, where the gas mixture equilibrates in temperature with lid 306. Gas then flows through resistively-permeable oxygenation tube 310, which is immersed in the culture medium (not shown) containing mitochondria, or eukaryotic cells containing mitochondria, in a cavity 312 of chamber 302 and also flows across a gaseous oxygen sensor 308. Chamber 302 contains a magnetic stirring device 314 and has a sensor 316 for measuring oxygen tension in the culture medium in cavity 312. The magnetic stirring device is configured both to circulate fluid from an exterior of oxygenation tube 310 to the cells, equalizing the oxygen tension throughout cavity 312, as well as maintaining cells or mitochondria in suspension. Tube 310 is resistively permeable in that, although oxygen can pass through its wall or membrane, oxygen passage through its wall occurs at a finite rate that depends on a gradient of oxygen partial pressure across its wall. While in tube 310 has a resistively permeable wall or membrane formed into a tubular shape, in alternative embodiments a resistively permeable wall or membrane may have a different shape.

A temperature control system, which in an embodiment includes a thermoelectric element 140, a temperature sensor, a feedback controller, and a power supply, is provided for heating or cooling the chamber 302 to an incubation temperature where living cells or mitochondria that may be within the chamber are biologically active. Gas supply 142 provides a determinable mixture of oxygen, nitrogen and carbon dioxide, and, in some embodiments, carbon monoxide or nitric oxide, along with water vapor, these gasses are provided to the chamber 302. Gas sensors may be provided for measurement of particular gasses in the mixture of gasses, and an oxygen sensor 308 is provided at a gas outlet of chamber 302.

Chamber 302 is also fitted with a dissolved-oxygen sensor 316 for measuring dissolved oxygen tension of fluid, such as a culture medium fluid, in chamber 302.

In an embodiment suitable for use with a cellular suspension, chamber 302 is a cell-suspension chamber 202 as illustrated in FIG. 2. In this embodiment, an inner quartz cylinder 204 holds a suspension of cells in a culture fluid. Quartz cylinder 204 is disposed within a cavity 205 within an aluminum block 206. A gas-permeable, thin-walled, small-bore silicone or Teflon® (trademark of E. I. du Pont de Nemours and Company, Wilmington, Del., for polytetrafluoroethylene) gas-permeable chamber-oxygenation tube 208, porous to small-molecule gasses, is wound about an axis into a coil within cylinder 204, and coupled to carry gas from a gas inlet 212 to a gas outlet 210. An outlet oxygen gas sensor 216, and/or and inlet oxygen gas sensor (not shown), are provided to measure oxygen concentrations in oxygenation tube 208. In an embodiment, gas inlet 212 and outlet 210 are formed as drilled passages in a stainless-steel cap 218 that mounts over cylinder 204 in block 206. In an embodiment, cap 218 has an axial hole filled with a stainless plug 220; removal of plug 220 provides access to an interior of cylinder 204 in chamber 205. In an embodiment, a removable access plug 225, which may be of Teflon or silicone, having a vent passage 227 that fills an axial hole in stainless plug 220, removal of plug 225 also provides access to an interior of cylinder 204 and vent passage 227 permits removal of air within chamber 205. A magnet encased in glass 222, serving as a magnetic stirring bar is placed within cylinder 204 to agitate cells or mitochondria into a suspension, and block 206 is mounted on a thermoelectric heating/cooling device 224, the heating/cooling device 224 in turn mounted on a heat-sink base 226. A hole 231 is drilled in block 206, and a hole 231 is drilled in cylinder 204, to permit oxygen sensor 230 to contact the cell suspension so sensor 230 can measure an oxygen tension in the culture fluid.

In an embodiment, a port is provided whereby a small quantity of additional culture medium containing a particular nutrient, or in alternative embodiments a particular poison, may be inserted into the chamber to permit determination of an effect of the nutrient or poison on the cells or mitochondria suspended in the chamber. In an embodiment, vent passage 227 is used for this purpose, in an alternative embodiment an additional passage (not shown) is provided to allow insertion of the additional culture medium.

In particular embodiments of particular use in spectrographic experiments, or in experiments involving cells capable of carrying out photosynthesis, holes (not shown) are drilled in block 206 to permit positioning of a spectrographic light transmitting coupler (not shown) behind quartz cylinder 204, and a received light coupler (not shown in FIG. 2) in front of quartz cylinder 204, such that an optical path exists along the axis of chamber oxygenation tube 208 coil; in FIG. 2 this path is perpendicular to the page. These couplers may couple light from an optical fiber into the chamber, and light from the chamber into an optical fiber. In other embodiments, light for photosynthetic reactions may be provided by light emitting diodes 250 (FIG. 3) or other light sources in a passage 248 at a base of the chamber. In embodiments, additional holes (not shown) may be drilled in block 206, which in particular embodiments may align with additional access holes in cylinder 204.

### Operating Modes

In all operating modes, if the cell-suspension chamber 202, 302, 270 is to be used, the chamber is filled with a biological material such as a suitable cell suspension in a culture medium, where the cells contain mitochondria, or a suitable suspension of separated mitochondria, gas sources 142 are adjusted to provide an appropriate gas mixture to chamber oxygenation tube 208, 310 and thermoelectric heater/cooler 224 is controlled by suitable electronics to provide suitable temperature to chamber 202, 302, 270.

All oxygen sensors 308, 316, 216, 230, are coupled to processor 126, where necessary, analog-to-digital converters are provided to allow digital processor 126 to read oxygen levels from the sensors.

Although permeable chamber oxygenation tube 208, 310 is permeable to oxygen, such that some oxygen passes between the lumen of the tube and surrounding culture medium, permeable chamber oxygenation tube 208, 310 resists oxygen flow through the tube walls. Since culture medium in cavity 205, 312, holds in solution an amount of oxygen that varies with oxygen tension, changes to oxygen concentration in permeable chamber oxygenation tube 208 propagate to the culture medium with a time constant dependent on the permeability of the tube. The permeability of the tube, and oxygen consumption rate of mitochondria, are also factors that affect oxygen concentration in the culture medium.

The subsystem of oxygenation tube 208, 310, and fluid-filled cavity 205, 312, therefore are roughly analogous to the electrical circuit of FIG. 4, with permeability of oxygenation tube 208, 310 wall corresponding roughly to conductance (an inverse of resistance) of resistor 502 and inductance of inductor 503 roughly corresponding to delay and inertia of oxygen dissolved in oxygenation tube 208, 310, walls. Dissolved oxygen in chamber cavity 205, 312 corresponding roughly to charge in capacitor 504, with oxygen consumption by mitochondria in the chamber corresponding roughly to current flow in current source 506, voltage 510 at input 508 corresponding roughly to oxygen partial pressure of the gas in the lumen of oxygenation tube 208, 310, and voltage 512 at an output node 514 corresponding roughly to oxygen partial pressure in culture medium inside the chamber cavity 205, 312.

The oxygen consumption (VO₂) by mitochondria in the chamber is calculated by processor 126 using machine readable instructions 130 in its memory for calculating oxygen consumption from data read from the oxygen sensors 308, 316, 230, 216. In an embodiment, processor 126 computes the oxygen consumption from:

$${K}_{s} = \frac{d {Pc O}_{2}}{d t} - {V O}_{2} + {K}_{p} ( f ( t ) ⊗ ( {Pt O}_{2} - {Pc O}_{2} ) ) \qquad Equation 1$$

Where PcO₂ is an oxygen partial pressure in the culture medium PtO₂ is an oxygen partial pressure of the gas mixture in tube lumen, f(t) is a time dependence of the gas system with the constraint that

$${\int }_{0}^{∞} f ( t ) = 1$$

and f(t)=0 for t<=0, Kₛ is a solubility of oxygen in the culture medium, [symbol] is the convolution operator and Kₚ is a lumped permeability constant of oxygen through the tubing wall. The constant Kₚ is dependent on the permeability constant of the tube material, the diameter, wall thickness and length of the tubing, and on the volume of the chamber.

Since many factors can alter oxygen flux in a biological material, it can be desirable to resolve time-dependent changes in the oxygen flux VO₂. Solving this equation for VO₂ permits the determination of the time-dependencies of VO₂. In the steady state, the oxygen consumption of the cells is given by:

VO₂=Kₚ(PtO₂−PcO₂) Equation 2

Any difference in calibration between the oxygen sensors 216 308 measuring oxygen partial pressure entering the tube and the oxygen sensor 230, 316 in the culture medium may be determined by allowing the system to reach a stable state with no mitochondria or other oxygen consuming substance in the chamber, processor 126 then determines a difference between the tube and culture medium oxygen sensor readings and corrects for the difference by adding this difference to following culture medium oxygen sensor readings. f(t) is determined by measuring oxygen partial pressure with sensors 216, 308 at the oxygenation tube when oxygen concentration of the gas is perturbed.

The constant Kₚ and function ƒ(t) can be calibrated by manipulating the oxygen tension within the chamber and performing a least fitting. The functional form of f(t) is parameterized, in one embodiment this function is ƒ(t)=kᵢ²te^{-k}^{ᵢ}ₜ for t>=0 and kᵢ is parameter. The oxygen tension in the chamber is raised from one steady state to a higher steady state and then returned to the first steady state ensuring that both steady states are in the saturating regime for oxygen tension such that the oxygen consumption does not change during this procedure. The oxygen consumption, Kₚ and Kᵢ can then be fitted to minimize χ², given by:

$${χ}^{2} = {( {K}_{s} \frac{d {Pc O}_{2}}{d t} + {V O}_{2} - {K}_{p} ( f ( t ) ⊗ ( {Pt O}_{2} - {Pc O}_{2} ) ) )}^{2} \qquad Equation 3$$

Kₛ of a particular culture medium and cell or mitochondria suspension may vary from that of water because of oxygen-absorbing substances like hemes, including heme-containing proteins like hemoglobin and myoglobin and some cytochromes, that may be present in the suspension, whether in the culture medium or the cells, and small volume differences between chambers, and is measured by assembling the system with tube, culture medium, and suspension, perturbing the oxygen concentration within the tube, and monitoring a time-dependent response of the oxygen concentration in the culture medium to the perturbed oxygen concentration.

Oxygen consumption by the cells in the suspension is measured continuously by measuring the oxygen tension at inlet, 212, and in the suspension with suspension oxygen sensor 230, 308. Oxygen consumption by suspended cells is then calculated from the difference in oxygen concentration and at least Kp.

Some eukaryotic cells not only have mitochondria, but also have chloroplasts. In an alternative embodiment having utility for measuring response of cells to illumination, light emitting devices are provided within the chamber, or optical fibers are providing for admitting light to the chamber. In these embodiments, the oxygen consumption calculated from the difference in oxygen concentration between the outlet 212 and the suspension oxygen sensor, may be negative. Negative oxygen consumption may provide a measure of oxygen generation by photosynthesis.

When the system of FIGS. 1-3 is used, an aqueous suspension of cells, or isolated mitochondria in fluid, is placed 602 in the chamber 205, 312. In some embodiments, the fluid is a culture medium. The chamber is then sealed 604 by, in a flat membrane embodiment adding membrane over the suspension and closing the chamber by attaching a chamber top. In the coiled-tubular-membrane embodiment of FIG. 2 or 3, the chamber is closed by inserting plug 220 and bleeding off gas at the suspension surface through passage 227; passage 227 may then be plugged.

Once the chamber is sealed, the system may be calibrated by changing 606 the oxygen concentration in gas flowing through the tube or across a flat membrane, while measuring oxygen tension in the fluid in the chamber. Constants are then fitted to the observed change in oxygen tension as previously described with reference to Equation 3. The chamber is then incubated 608 while observing oxygen tension in the gas and in the fluid. An oxygen consumption for the biological material by computing a difference between the oxygen tension in gas and fluid, and multiplying 610 by a constant determined by the fitting operation. The system may then be perturbed 612 to determine an effect of adding a nutrient or poison, and a perturbed-system oxygen consumption for the biological material computed by computing a difference between the oxygen tension in gas and fluid, and multiplying 614 by the constant determined by the fitting operation.

The system and method herein described can be assembled with various combinations of the concepts described herein, as outlined in following paragraphs.

In an embodiment designated A, a system for measuring oxygen consumption in mitochondria has a chamber adapted to contain a culture medium containing a biological material incorporating mitochondria, which may or may not be in cells, a resistively gas-permeable membrane within the chamber having a first side positioned to contact culture medium present in the chamber, a first sensor positioned to measure oxygen tension in the culture medium, a second sensor positioned to measure oxygen in gas adjacent a second side of the membrane, and a processor coupled to read data from the first and the second sensor, the processor having a memory having recorded therein machine readable instructions for determining oxygen consumption by the biological material from the data.

In an embodiment designated AA, of the embodiment designated A wherein the membrane is formed into a tube, and wherein the gas adjacent a second side of the membrane is within a lumen of the tube.

In an embodiment designated AB, of the embodiment designated A or AA wherein the membrane is made from a polymer selected from the group consisting of polytetrafluoroethylene and silicone.

In an embodiment designated AC, of the embodiment designated A, AA, or AB, further including a magnetic stirring apparatus for maintaining the biological material in suspension.

In an embodiment designated AD, of the embodiment designated A, AA, AB, or AC, wherein the machine readable instructions for determining oxygen consumption by the biological material comprises instructions for using an oxygen permeability constant of the membrane and a difference between the oxygen concentration of the fluid and gas oxygen concentration to calculate oxygen passage across the membrane.

In an embodiment designated Ae, of the embodiment designated A, AA, AB, AC, or AD wherein, wherein the machine readable instructions for determining oxygen consumption by the biological material include instructions for multiplying a constant by a difference between an oxygen concentration derived from measurements by the first sensor and an oxygen concentration derived from measurements by the second sensor.

A method designated B of measuring oxygen flux of a biological material including: placing the material, with a fluid, in a chamber; exposing the fluid to a first side of a resistively-permeable membrane; exposing a second side of the resistively-permeable membrane to a gas mixture, the gas mixture containing oxygen at a gas oxygen tension; measuring an oxygen partial pressure of the fluid; and calculating the oxygen flux of the biological material from an oxygen permeability constant of the membrane and a difference between the oxygen concentration of the fluid and gas oxygen concentration.

A method designated BA including the method designated B wherein the membrane is formed into a tube and the gas mixture flows from a gas source through a lumen of the tube.

A method designated BB including the method designated B or BA, wherein the oxygen flux is further calculated using a solubility of oxygen in the fluid and further comprising calibrating the solubility of oxygen in the fluid by changing an oxygen concentration of the gas mixture and measuring a response of oxygen concentration of the fluid.

A method designated BC including the method designated B, BA or BC wherein the oxygen flux is calculated by steps including multiplying a constant by a difference between an oxygen concentration derived from measurements made by the first sensor and an oxygen concentration derived from measurements made by the second sensor.

A system designated C for measuring oxygen consumption in biological materials including: a chamber adapted to contain a culture medium containing biological material; a resistively gas-permeable membrane within the chamber having a first side positioned to contact culture medium present in the chamber; a first sensor positioned to measure oxygen tension in the culture medium; a second sensor positioned to measure oxygen in gas adjacent a second side of the membrane; a processor coupled to read data from the first and the second sensor, the processor having a memory having recorded therein machine readable instructions for determining oxygen consumption by the biological material from the data.

A system designated CA including the system designated C wherein machine readable instructions for determining oxygen consumption by the biological material include instructions for multiplying a constant by oxygen tension datum determined from the first sensor and an oxygen tension datum determined from the second sensor.

Those skilled in the art will appreciate that the presently disclosed instrumentalities teach by way of example and not by limitation. Therefore, the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall there between.

1. A system for measuring oxygen consumption in mitochondria comprising:
a chamber adapted to contain a culture medium containing a biological material comprising mitochondria;
a resistively gas-permeable membrane within the chamber having a first side positioned to contact culture medium present in the chamber;
a first sensor positioned to measure oxygen tension in the culture medium;
a second sensor positioned to measure oxygen in gas adjacent a second side of the membrane;
a processor coupled to read data from the first and the second sensor, the processor having a memory having recorded therein machine readable instructions for determining oxygen consumption by the biological material from the data.

2. The system of claim 5 wherein the membrane is formed into a tube, and wherein the gas adjacent a second side of the membrane is within a lumen of the tube.

3. The system of claim 2 wherein the membrane comprises a polymer selected from the group consisting of polytetrafluoroethylene and silicone.

4. The system of claim 5 further comprising a magnetic stirring apparatus for maintaining the biological material in suspension.

5. The system of claim 1 wherein the machine readable instructions for determining oxygen consumption by the biological material comprises instructions for using an oxygen permeability constant of the membrane and a difference between the oxygen concentration of the fluid and gas oxygen concentration to calculate oxygen passage across the membrane.

6. The system of claim 5, wherein the machine readable instructions for determining oxygen consumption by the biological material include instructions for multiplying a constant by a difference between an oxygen concentration derived from measurements by the first sensor and an oxygen concentration derived from measurements by the second sensor.

7. A method of measuring oxygen flux of a biological material comprising:
placing the material, with a fluid, in a chamber;
exposing the fluid to a first side of a resistively-permeable membrane;
exposing a second side of the resistively-permeable membrane to a gas mixture, the gas mixture containing oxygen at a gas oxygen tension;
measuring an oxygen partial pressure of the fluid; and
calculating the oxygen flux of the biological material from an oxygen permeability constant of the membrane and a difference between the oxygen concentration of the fluid and gas oxygen concentration.

8. The method of claim 7 wherein the membrane is formed into a tube and the gas mixture flows from a gas source through a lumen of the tube.

9. The method of claim 8 wherein the oxygen flux is further calculated using a solubility of oxygen in the fluid and further comprising calibrating the solubility of oxygen in the fluid by changing an oxygen concentration of the gas mixture and measuring a response of oxygen concentration of the fluid.

10. The method of claim 9 wherein the oxygen flux is calculated by steps including multiplying a constant by a difference between an oxygen concentration derived from measurements made by the first sensor and an oxygen concentration derived from measurements made by the second sensor.

11. A system for measuring oxygen consumption in biological materials comprising:
a chamber adapted to contain a culture medium containing biological material;
a resistively gas-permeable membrane within the chamber having a first side positioned to contact culture medium present in the chamber;
a first sensor positioned to measure oxygen tension in the culture medium;
a second sensor positioned to measure oxygen in gas adjacent a second side of the membrane;
a processor coupled to read data from the first and the second sensor, the processor having a memory having recorded therein machine readable instructions for determining oxygen consumption by the biological material from the data.

12. The system of claim 11 wherein machine readable instructions for determining oxygen consumption by the biological material include instructions for multiplying a constant by oxygen tension datum determined from the first sensor and an oxygen tension datum determined from the second sensor.
